(19) United States
(12) Patent Application Publication
(10) Pub. No.: US 2026/0215384 A1
(43) Pub. Date: Jul. 30, 2026

(54) AcTEAvator System

(71) Applicant: The Regents of the University of California, Oakland, CA (US)

(72) Inventors: Jacquelyn Gervay-Hague, Davis, CA (US); David M. Tricoli, Davis, CA (US)

(21) Appl. No.: 19/144,905

(22) PCT Filed: Dec. 22, 2023
(86) PCT No.: PCT/US2023/085747

§ 371 Date: Jun. 30, 2025

Related U.S. Application Data
(60) Provisional application No. 63/477,711, filed on Dec. 29, 2022.

(51) Int. Cl. A01H 4/00; A01G 2/10; A01G 22/60; A61L 2/18; A61L 101/06; A61L 103/05

(52) U.S. Cl. CPC A01H 4/005; A01G 2/10; A01G 22/60; A61L 2/18; A61L 2101/06; A61L 2103/05

(57) ABSTRACT

Provided herein are methods and kits for propagation of tea plant varieties that can reduce the time to harvest from about 7 years to about 5 months by using a sequence of media formulations comprising gibberellic acid (GA₃), indole-3-butyric acid (IBA), and at least one of benzylaminopurine (BAP) and meta-Topolin (mT).

### CROSS-REFERENCE TO RELATED APPLICATION

This application claims priority to U.S. Provisional Application No. 63/477,711, filed Dec. 29, 2022, the disclosure of which is hereby incorporated by reference in its entirety for all purposes.

### STATEMENT REGARDING FEDERALLY SPONSORED RESEARCH OR DEVELOPMENT

This invention was made with Government support under Grant No. 1758530, awarded by the National Science Foundation (NSF). The Government has certain rights in the invention.

### BACKGROUND

Tea is the most widely consumed non-alcoholic beverage in the world after water. Yet cultivation of tea has many challenges. Tea plants require long cultivation periods. Harvesting and processing tea is labor intensive. Additionally, tea production typically relies upon international transport, which leaves a large carbon footprint.

Annual global tea production is about 6 million metric tons (MT), creating approximately 12 million MT in CO₂ emissions in the countries of production and additional CO₂ emissions during its export. About ⅓ of tea production is exported to non-tea growing countries such as Russia and the United States. For example, most of the commodity tea coming into the U.S. is shipped from ports in India, China, and Japan. The average length of the major import routes for tea into the U.S. is about 27,000 kilometers, resulting in an estimated U.S. carbon footprint for tea importation that is >100,000 MT in CO₂ emissions annually.

Camellia sinensis, the plant from which tea is extracted, is an evergreen woody plant. It takes approximately 2 years to propagate liners (young plants) from mother stock and an additional 5 years of field growth before reaching a steady production potential of approximately 0.5 lb dried tea per plant per year. In the USA, it costs about $100,000 to establish an acre containing 3600 plants, not including land costs. One acre of mature tea plants (planted for 5+ years) yields about 1 MT of dried tea per year, with a retail value of approximately $40,000.

High quality tea is made from the terminal buds and two young leaves (“one tip, two leaves”) of the Camellia sinensis plant. During harvesting season, tea flush, or the plucking of a tea plant's harvest, is performed by hand every 3-4 weeks. Some have employed motor driven field hedgers in harvesting but harvesting with hedgers does not provide reproducible tea quality and often damages tea leaves. Moreover, hedgers are fueled by gasoline, which contributes to further CO₂ emissions when widely adopted. Since tea leaves begin to oxidize soon after harvesting, tea processing, which varies by the type of tea being processed, is also done very close to the site of tea harvesting to maintain quality.

Although domestic U.S. tea production was first recorded to occur in the 1700s, it was not until 2003 that the first commercial tea plantation was established in Charleston, South Carolina. Fewer than 100 acres of tea are planted in the U.S. today. Labor costs associated with tea processing are considered a major barrier to establishing US tea growth and production. (D'Auria et al., 2022).

Micropropagation of Camellia sinensis is one method for generating large numbers of tea plants in a short period of time (Mukhopadyay, M. et al., 2016). However, shoot organogenesis is relatively difficult in Camellia species, especially in the vegetative parts (Frisch, C. H, and Camper, N. D., 1987; Gunasekare and Evans, 2000).

This application describes a system for propagating tea plants in controlled environments that optimize shoot proliferation and shoot elongation. The methods and kits described herein have the potential to reduce time to harvest for new tea plants from about 7 years to about 5 months. This would enable large-scale domestic tea production in the near future from varieties with desirable traits, curtailing the carbon footprint associated with the import of tea from abroad.

### BRIEF SUMMARY OF THE INVENTION

In a first aspect, this invention provides a method of propagating a Camellia plant, comprising: (i) placing the base of a shoot cutting from a Camellia plant in a budding medium for a sufficient length of time until the shoot cutting has at least one bud that has elongated to at least 1 cm, wherein the budding medium comprises plant tissue culture medium such as MS medium with half-strength macronutrients and full-strength micronutrients or its equivalent, a gibberellin, an auxin, and a cytokinin; (ii) transferring the shoot cutting with at least one bud to a shoot multiplication medium for a sufficient length of time until the shoot cutting has at least two offshoots, wherein the shoot multiplication medium comprises plant tissue culture medium such as DKW medium or its equivalent, a gibberellin, an auxin, and a cytokinin; and (iii) transferring the shoot cutting with at least two offshoots to a shoot elongation medium comprising plant tissue culture medium such as DKW medium or its equivalent, a gibberellin, an auxin, and a cytokinin.

In some embodiments, the shoot cutting is 3 cm to 6 cm long before (i). In some embodiments, the method further comprises pretreating the shoot cutting before (i) by surface sterilizing the shoot cutting in sodium hypochlorite solution containing Tween 20 followed by rinsing in sterile distilled water and removing the base of the shoot cutting. In some embodiments, the sodium hypochlorite solution is in the concentration range of about 0.5 to 4% (v/v), and the treatment time is 10 to 30 minutes. In some embodiments, the sodium hypochlorite solution is in the concentration range of about 0.7 to 1.7% (v/v), and the treatment time is 15 to 20 minutes.

In some embodiments, the gibberellin in the budding medium comprises gibberellic acid (GA₃). In some embodiments, the auxin in the budding medium comprises indole-3-butyric acid (IBA). In some embodiments, the cytokinin in the budding medium is at a concentration in the range of 0.1 to 50 mg/L. In some embodiments, the auxin in the budding medium is at a concentration of 0 to 10 mg/L. In some embodiments, the cytokinin in the budding medium comprises less than 3 mg/L aBAP). In some embodiments, the cytokinin in the budding medium comprises 1 mg/L BAP.

In some embodiments, the budding medium comprises ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and plant preservative mixture (PPM™) at a concentration in the range of 0 to 10 mL/L.

In some embodiments, the budding medium comprises ascorbic acid at a concentration in the range of 50 to 150 mg/L, citric acid at a concentration in the range of 100 to 200 mg/L, L-cysteine at a concentration in the range of 50 to 150 mg/L, reduced glutathione at a concentration in the range of 25 to 35 mg/L, glutamine at a concentration in the range of 250 to 350 mg/L, putrescine at a concentration in the range of 0.5 to 1.5 mM, ferrous sulfate at a concentration in the range of 100 to 150 mg/L, BAP at a concentration in the range of 0.5 to 1.5 mg/L, GA₃ at a concentration in the range of 0 to 0.5 mg/L, IBA at a concentration in the range of 0 to 0.5 mg/L, and PPM™ at a concentration in the range of 0 to 0.5 mL/L.

In some embodiments, the budding medium comprises MS medium with half-strength macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, ferrous sulfate, 1 mg/L BAP, 0.1 mg/L GA₃, 0.1 mg/L IBA, 5 mg/L chlorophenol red, and 0.1 mL/L PPM™.

In some embodiments, the bud is discernible as the emergence of new growth. In some embodiments, the bud has elongated to approximately 2-3 cm after (i).

In some embodiments, the cytokinin in the shoot multiplication medium is at a concentration in the range of 0.1 to 50 mg/L. In some embodiments, the auxin in the shoot multiplication medium is at a concentration of 0 to 10 mg/L. In some embodiments, the gibberellin in the shoot multiplication medium comprises GA₃. In some embodiments, the auxin in the shoot multiplication medium comprises IBA. In some embodiments, the cytokinin in the shoot multiplication medium comprises less than 3 mg/L BAP. In some embodiments, the cytokinin in the shoot multiplication comprises 1 mg/L BAP.

In some embodiments, the shoot multiplication medium comprises DKW medium, sucrose at a concentration in the range of 10 to 50 g/L, putrescine at a concentration in the range of 0.1 to 10 mM, glutamine at a concentration in the range of 0 to 500 mg/L, BAP is at a concentration in the range of 0.1 to 50 mg/L, GA₃ at a concentration in the range of 0 to 40 mg/L, and IBA at a concentration in the range of 0 to 10 mg/L.

In some embodiments, the shoot multiplication medium comprises DKW medium, sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0.5 to 1.5 mg/L, GA₃ at a concentration in the range of 0 to 0.5 mg/L, and IBA at a concentration in the range of 0 to 0.5 mg/L.

In some embodiments, the shoot multiplication medium comprises DKW medium, 20 g/L sucrose, 1 mM putrescine, 300 mg/L glutamine, 1 mg/L BAP, 0.1 mg/L GA₃, and 0.1 mg/L IBA.

In some embodiments, the shoot cutting has at least three offshoots from lateral bud expansion after (ii).

In some embodiments, the cytokinin in the shoot elongation medium is at a concentration in the range of 0.1 to 50 mg/L. In some embodiments, the auxin in the shoot elongation medium is at a concentration of 0 to 10 mg/L. In some embodiments, the gibberellin in the shoot elongation medium comprises GA₃. In some embodiments, the auxin in the shoot elongation medium comprises IBA. In some embodiments, the cytokinin in the shoot elongation medium comprises meta-Topolin (mT) or BAP. In some embodiments, the shoot elongation medium comprises 3 mg/L mT or 0.1 mg/L BAP.

In some embodiments, the shoot elongation medium comprises sucrose in the concentration range of 10 to 50 g/L, putrescine in the concentration range of 0.1 to 10 mM, glutamine in the concentration range of 0 to 500 mg/L, BAP at a concentration in the range of 0.1 to 50 mg/L or mT in the concentration range of 0.1 to 10 mg/L, GA₃ in the concentration range of 0 to 40 mg/L, and IBA in the concentration range of 0 to 10 mg/L.

In some embodiments, the shoot elongation medium comprises sucrose in the concentration range of 25 to 35 g/L, putrescine in the concentration range of 0.5 to 1.5 mM, glutamine in the concentration range of 250 to 350 mg/L, BAP at a concentration in the range of 0 to 0.5 mg/L or mT in the concentration range of 2.5 to 3.5 mg/L, GA₃ in the concentration range of 0 to 0.5 mg/L, and IBA in the concentration range of 0 to 0.5 mg/L.

In some embodiments, the shoot elongation medium comprises DKW medium, 20 g/L sucrose, 1 mM putrescine, 300 mg/L glutamine, 0.1 mg/L BAP or 3 mg/L mT, 0.1 mg/L GA₃, and 0.1 mg/L IBA.

In some embodiments, the method further comprises (iv) transferring a shoot cutting with at least three offshoots after (iii) into optimized culture medium comprising DKW medium and a gibberellin, an auxin, and a cytokinin mT.

In some embodiments, the cytokinin in the optimized culture medium is at a concentration in the range of 0.1 to 50 mg/L. In some embodiments, the auxin in the optimized culture medium is at a concentration of 0 to 10 mg/L. In some embodiments, the gibberellin in optimized culture medium comprises GA₃. In some embodiments, the auxin in optimized culture medium comprises IBA. In some embodiments, the cytokinin mT in the optimized culture medium comprises 3 mg/L mT.

In some embodiments, the optimized culture medium comprises DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, and sodium thiosulfate and sodium nitrate at a concentration in the range of 5 to 20 mM.

In some embodiments, the optimized culture medium comprises DKW medium, sucrose at a concentration in the range of 25 to 35 g/L, mT at a concentration in the range of 2.5 to 3.5 mg/L, GA₃ at a concentration in the range of 0 to 0.5 mg/L, IBA at a concentration in the range of 0 to 0.5 mg/L, calcium gluconate at a concentration in the range of 0.8 to 1.8 g/L, putrescine at a concentration in the range of 0.5 to 1.5 mM, glutamine at a concentration in the range of 250 to 350 mg/L, and sodium thiosulfate and sodium nitrate at a concentration in the range of 10 to 15 mM.

In some embodiments, optimized culture medium comprises DKW medium, 30 g/L sucrose, 0.1 mg/L GA₃, 1 mg/L IBA, 3 mg/L mT, 1.3 g/L calcium gluconate, 1 mM putrescine, 300 mg/L glutamine, and 12 mM sodium thiosulfate and silver nitrate.

In some embodiments, the shoot cutting is at least 2 cm longer after (iv) than it was before (iv). In some embodiments, the shoot cutting is at least 4 cm longer after (iv) than it was before (iv).

In some embodiments of the method, upon the shoot cutting in step (iv) of the method develops into a juvenile plant that is at least two inches tall (e.g., at least two, three, four, five, six, seven, eight, nine, or ten inches tall), has at least five leaves (e.g., at least five, six, seven, eight, nine, or ten leaves), and/or has at least one offshoot (e.g., at least one, two, three, four, or five offshoots), a step (v) of harvesting a top bud and a first set of leaves (e.g., at least two, three, four, five, six, seven, eight, nine, or ten leaves) from each offshoot of the plant is performed. In some embodiments, all of the leaves of the plant (e.g., a juvenile plant or a full grown mature plant) is harvested.

In some embodiments of the method, upon the shoot cutting in step (iv) of the method develops into a full grown mature plant that is at least eight inches tall (e.g., at least eight, nine, or ten inches tall), has at least ten leaves (e.g., at least ten, twelve, thirteen, fourteen, fifteen, sixteen, seventeen, eighteen, nineteen, or twenty leaves), and/or has at least two offshoots (e.g., at least two, three, four, or five offshoots), a step (v) of harvesting a top bud and a first set of leaves (e.g., at least two, three, four, five, six, seven, eight, nine, or ten leaves) from each offshoot of the plant is performed. In some embodiments, all of the leaves of the full grown mature plant is harvested.

In certain embodiments, the time between the step of placing the base of a shoot cutting from the Camellia plant in the budding medium to harvesting the leaves of the plant is about two, three, four, five, or six months. In certain embodiments, the time between the step of placing the base of a shoot cutting from the Camellia plant in the budding medium to harvesting the leaves of the plant is about five months.

In some embodiments, the method further comprises rooting the shoot cutting before placing it into soil. In some embodiments, the sufficient length of time in (i) and/or (ii) is at least two intervals, wherein each interval is about 4 weeks. In some embodiments, the shoot cutting is placed in fresh medium after each interval.

In some embodiments, the shoot cutting with at least three offshoots is sub-cultured three times at an interval of 4 weeks into fresh optimized culture medium.

In some embodiments, the Camellia plant is a tea plant (Camellia sinensis). In some embodiments, the tea plant is one of Benikori, Bohea, UC-K02, UC-K03, UC-K04, UC-N02, UC-N06, UC-N08, UC-N09, UC-N15, Yabukita, and Yutaka Midori plant varieties. In some embodiments, the tea plant variety is UC-K03 or UC-K04.

In another aspect, the present disclosure describes a kit for propagation of a tea plant comprising: a budding medium comprising ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L; a shoot multiplication medium comprising DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L; a shoot elongation medium comprising DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or mT at a concentration in the range of 0.1 to 10 mg/L, GA₃ at a concentration in the range of 0 to 40 mg/L, and IBA at a concentration in the range of 0 to 10 mg/L; and an optimized culture medium comprising DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

In some embodiments, the kit for propagation of a tea plant comprises: a budding medium comprising ascorbic acid at a concentration in the range of 50 to 150 mg/L, citric acid at a concentration in the range of 100 to 200 mg/L, L-cysteine at a concentration in the range of 50 to 150 mg/L, reduced glutathione at a concentration in the range of 25 to 35 mg/L, glutamine at a concentration in the range of 250 to 350 mg/L, putrescine at a concentration in the range of 0.5 to 1.5 mM, ferrous sulfate at a concentration in the range of 100 to 150 mg/L, BAP at a concentration in the range of 0.5 to 1.5 mg/L, GA₃ at a concentration in the range of 0 to 0.5 mg/L, IBA at a concentration in the range of 0 to 0.5 mg/L, and PPM™ at a concentration in the range of 0 to 0.5 mL/L; a shoot multiplication medium comprising sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0.5 to 1.5 mg/L, GA₃ at a concentration in the range of 0 to 0.5 mg/L, and IBA at a concentration in the range of 0 to 0.5 mg/L; a shoot elongation medium comprising sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0 to 0.5 mg/L or mT at a concentration in the range of 2.5 to 3.5 mg/L, GA₃ at a concentration in the range of 0 to 0.5 mg/L, IBA at a concentration in the range of 0 to 0.5 mg/L; and an optimized culture medium comprising sucrose at a concentration in the range of 25 to 35 g/L, mT at a concentration in the range of 2.5 to 3.5 mg/L, GA₃ at a concentration in the range of 0 to 0.5 mg/L, IBA at a concentration in the range of 0 to 0.5 mg/L, calcium gluconate at a concentration in the range of 0.8 to 1.8 g/L, putrescine at a concentration in the range of 0.5 to 1.5 mM, glutamine at a concentration in the range of 250 to 350 mg/L, and sodium thiosulfate and sodium nitrate at a concentration in the range of 10 to 15 mM.

In some embodiments, the kit for propagation of a tea plant comprises: a budding medium comprising MS medium with half-strength macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, ferrous sulfate, 1 mg/L BAP, 0.1 mg/L GA₃, 0.1 mg/L IBA, 5 mg/L chlorophenol red, and 4 mL/L PPM™, a shoot multiplication medium comprising DKW medium, 20 g/L sucrose, 1 mM putrescine, 300 mg/L glutamine, 1 mg/L BAP, 0.1 GA₃, and 0.1 mg/L IBA; a shoot elongation medium comprising DKW medium, 20 g/L sucrose, 1 mM putrescine, 300 mg/L glutamine, 0.1 mg/L BAP or 3 mg/L mT, 0.1 mg/L GA₃, and 0.1 mg/L IBA; and an optimized culture medium comprising DKW medium, 30 g/L sucrose, 3 mg/L mT, 0.1 mg/L GA₃, 1 mg/L IBA, 1.3 g/L calcium gluconate, 1 mM putrescine, 300 mg/L glutamine, and 1 ml/L of 12 mM sodium thiosulfate and silver nitrate.

In another aspect, the disclosure provides a composition comprising a budding medium for propagation of a tea plant, wherein the budding medium comprises ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L.

In another aspect, the disclosure provides a composition comprising a shoot multiplication medium for propagation of a tea plant, wherein the shoot multiplication medium comprises DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L.

In another aspect, the disclosure provides a composition comprising a shoot elongation medium for propagation of a tea plant, wherein the shoot elongation medium comprises DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or cytokinin at a concentration in the range of 0.1 to 10 mg/L, GA₃ at a concentration in the range of 0 to 40 mg/L, and IBA at a concentration in the range of 0 to 10 mg/L.

In another aspect, the disclosure provides a composition comprising an optimized culture medium for propagation of a tea plant, wherein the optimized culture medium comprises DKW medium, sucrose at a concentration in the range of 10 to 50 g/L, cytokinin at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

In another aspect, the disclosure provides a Camellia plant produced by the method described herein. The disclosure also provides a Camellia leaf harvested from such a plant produced by the method described herein.

### BRIEF DESCRIPTION OF THE DRAWINGS

FIG. 1 depicts the increases in shoot length after three four-week subcultures for 12 different varieties of tea after growth in optimized culture medium. Mean value (bars) and standard deviations (error bars), n=12. The same letters indicate no statistically significant differences among varieties (p<0.05) using Duncan's test.

FIG. 2 shows the shoot number after three four-week subcultures for 12 different varieties of tea after growth in optimized culture medium. Mean values (bars) and standard deviations (error bars), n=12. The same letters indicate no statistically significant differences among varieties (p<0.05) using Duncan's test.

FIG. 3 depicts the correlation between shoot length and number of shoots after three four-week subcultures for 12 different varieties of tea in the optimized culture media.

FIG. 4 shows dendrograms of tea plant varieties, including UC-K03 and UC-K04, generated using neighbor-joining algorithms with 100 bootstrap replicates.

FIGS. 5A and 5B show micropropagation of Benikori tea after 8 weeks in RITA bioreactor on DKW minimal organics medium supplemented with 1.0 mM putrescine, 300 mg/L L-glutamine, 0.1 mg/L GA₃, 0.1 mg/L IBA and 3 mg/L meta-topolin (FIG. 5A) or 3 mg/L BA (FIG. 5B). Tissue was immersed in liquid medium for 2 minutes, once per day.

FIGS. 5C and 5D show micropropagation of Benikori tea after 12 weeks in RITA bioreactor on DKW minimal organics medium supplemented with 1.0 mM putrescine, 300 mg/L L-glutamine, 0.1 mg/L GA₃, 0.1 mg/L IBA and 3 mg/L meta-topolin (FIG. 5C) or 3 mg/L BA (FIG. 5D). Tissue was immersed in liquid medium for 2 minutes, once per day.

FIG. 5E shows weight increases of the plants in FIG. 5C and FIG. 5D.

FIG. 6 shows examples of juvenile plants.

FIG. 7 shows an example of a schematic illustrating the use of the system to continually produce biomaterial in vitro.

### DETAILED DESCRIPTION OF THE INVENTION

### I. Introduction

Even though there is interest in introducing new tea plant cultivars with desirable traits such as tolerance to biotic or abiotic stress, high yield, and high quality of tea product, conventional breeding of tea plants is a very slow process. An alternative to conventional breeding is micropropagation in a controlled environment, which is particularly useful for generating large numbers of plants within a short period of time.

However, shoot organogenesis from vegetative parts is relatively difficult in Camellia species (Frish and Camper 1987; Gunasekare, M. T. K. and Evans, P. K. 2000). Furthermore, shoot organogenesis techniques vary by plant type and traditional techniques require the use of disinfectants, varying concentrations of phytohormones, and sometimes different antibiotics, antifungals, and antivirals for each stage of the growth and development process (Liang, C. et al., 2019). Also, phytohormones cannot necessarily substitute for each other, even if they belong to the same class of phytohormones.

For example, Kucharska, D. et al. (2020) found that application of the cytokinin mT is superior to the cytokinin BAP for micropropogation of gooseberry plants. Whereas BAP causes a tendency for necrosis and a lack of shoot elongation in gooseberry plants, mT increases mean shoot number and reduces necrosis to almost zero. Meanwhile, the auxins indole-3-acetic acid (IAA) and IBA in combination with mT have no effect on shoot number in gooseberry plants but cause a tendency for necrosis in some genotypes.

The effects of different combinations and concentrations of BAP, the cytokinin thidiazuron (TDZ), and GA₃ on shoot multiplication and elongation of micropropagated tea plants have been studied previously by Gonbad, R. A. et al. (2014). In those studies, a combination of BAP and GA₃ could be effective in stimulating the simultaneous multiplication and elongation of shoots, whereas TDZ caused hyperhydricity, a physiological malformation that results in excessive hydration. Also, GA₃ showed no difference in the average number of shoots produced, but shoot elongation was enhanced when GA₃ was included in the medium.

The number of shoots produced in those studies was significantly affected by the different concentrations of BAP used. As expected, medium without BAP produced no shoots, since it is necessary for multiplication, but increasing the BAP concentration from 5 to 7 mg/L also reduced the number of shoots produced due to shoot necrosis and fasciation. Id. The highest average number of shoots reported by Gonbad, R. A. et al. (2014) was obtained in a medium supplemented with 3 mg/L BAP.

Described herein is a method that can be used for efficient multiplication and elongation of micropropagated C. sinensis plants and a kit that can be used to carry out this method. We show that this method works particularly well for the UC-K03 and UC-K04 cultivars.

In contrast to Gonbad, R. A. et al. (2014), we found that for the tea varieties tested in our micropropagation studies, BAP inhibits growth at a concentration of 3 mg/L. Therefore, 1 mg/L BAP was used to optimize tea plant growth. Also, mT had not been previously used in the micropropagation of tea plants. Here, we find that the combination of mT, GA₃, and IBA in optimized culture medium was able to provide shoot multiplication and elongation at the same time, which should reduce production time.

### II. Definitions

As used herein, the following terms have the meanings ascribed to them unless specified otherwise.

The term “about” denotes a range of +/−10% of a pre-determined value. For example, “about 10” sets a range of 90% to 110% of 10, i.e., 9 to 11.

“Murashige and Skoog medium” (MS medium) and “Driver and Kuniyuki Walnut medium” (DKW medium) are commercially available plant growth media that contain macronutrients and micronutrients. Murashige, T. and Skoog, F. (1962); Driver, J. A. and Kuniyuki, A. H. (1984). They are typically supplemented with sugars, vitamins, amino acids (e.g., putrescine which is used in the first step in polyamine synthesis), and biocides or fungicides such as Plant Preservative Mixture (PPM™) when used for plant tissue culture. They are also often combined with gelling agents such as agar for plant growth on semi-solid media. MS medium and DKW medium with minimal organics each have known compositions. Other plant growth media that are equivalent MS medium and DKW medium in terms of providing basic nutrients are available or can be synthesized. See, e.g., Oseni, O. M. (2018). The macronutrients and micronutrients in MS medium can be prepared as full-strength or half-strength preparations. In the latter, the quantity of macronutrients and/or micronutrients is halved within a given volume.

Plant growth media is also typically supplemented with phytohormones that are required for or enhance plant growth and development. These may include auxins, cytokinins, gibberelins, abscisic acid, and ethylene.

“Auxins” are responsible for cell elongation in phototropism and gravitropism as well as meristem differentiation, which controls leaf development and arrangement. Apical dominance, flowering, fruit setting, and ripening are all under the direct or indirect control of auxins. Indole-3-acetic acid (IAA) and indole butyric acid (IBA) are naturally occurring auxins, but synthetic auxins such as naphthalene acetic acid (NAA) and 2,4-dichlorophenoxyacetic acid (2,4-D) have also been developed.

“Cytokinins” promote cell division and are therefore abundant in actively growing tissues such as roots. They play a role in differentiation of roots and shoots as well as the timing of leaf senescence. Naturally occurring cytokinins include trans-zeatin (tZ), isopentyladenine (iP), dihydrozeatin, kinetin, cis-zeatin, and meta-Topolin (mT). Synthetic cytokinins include a synthetic version of kinetin, as well as 6-benzylaminopurine (BAP, also known as benzyl adenine or BA). Analogs of cytokinins such as thidiazuron (N-phenyl-N′-1,2,3-thiadiazol-5-ylurea, TDZ) are also used commercially to inhibit leaf yellowing.

“Gibberellins” stimulate shoot elongation, seed germination, and the maturation of fruit and flowers and are often located within actively growing parts of plants such as roots, young leaves, and seed embryos. Over 100 structurally related gibberellins are known. GA₃, or gibberellic acid, is involved in multiple developmental processes, including seed germination, flowering, and fruit development.

### III. Description of the Embodiments

### Methods for Propagating a Camellia Plant

In a first aspect, the present invention provides methods of propagating a Camellia plant. More particularly, the invention provides a method comprising: (i) placing the base of a shoot cutting from a Camellia plant in a budding medium for a sufficient length of time until the shoot cutting has at least one bud that has elongated to at least 1 cm, wherein the budding medium comprises plant tissue culture medium such as MS medium with half-strength macronutrients and full-strength micronutrients or its equivalent, a gibberellin, an auxin, and a cytokinin; (ii) transferring the shoot cutting with at least one bud to a shoot multiplication medium for a sufficient length of time until the shoot cutting has at least two offshoots, wherein the shoot multiplication medium comprises plant tissue culture medium such as DKW medium or its equivalent, a gibberellin, an auxin, and a cytokinin; and (iii) transferring the shoot cutting with at least two offshoots to a shoot elongation medium comprising plant tissue culture medium such as DKW medium or its equivalent, a gibberellin, an auxin, and a cytokinin. In some embodiments, the cytokinin is at a concentration in the range of 0.1 to 50 mg/L. In some embodiments, the auxin is at a concentration of 0 to 10 mg/L.

### A. Shoot Cutting

A “shoot cutting” is a portion of a plant that is severed from the rest of the plant and induced to form roots and/or shoots, thus producing a new plant. In some embodiments, the shoot cutting is 3 cm to 6 cm long before (i). In some embodiments, the method comprises pretreating the shoot cutting before (i) by surface sterilizing the shoot cutting in sodium hypochlorite solution containing Tween 20 followed by rinsing in sterile distilled water and removing the base of the shoot cutting. In some embodiments, the sodium hypochlorite solution is at a concentration in the range of 0.5% to 4% (v/v). In some embodiments, the sodium hypochlorite solution is at a concentration in the range of 0.7% to 1.7%. In some embodiments, the surface sterilization time is in the range of 10 min to 30 min. In some embodiments, the surface sterilization time is in the range of 15 min to 25 min. In some embodiments, surface sterilizing the shoot cutting comprises placing the shoot cutting in a 1.2% (v/v) sodium hypochlorite solution containing 5 mL Tween 20 for 20 min followed by three rinses in sterile distilled water.

### B. Shoot Budding

In some embodiments, the gibberellin in the budding medium comprises gibberellic acid (GA₃). In some embodiments, the auxin in the budding medium comprises indole-3-butyric acid (IBA). In some embodiments, the cytokinin in the budding medium is at a concentration in the range of 0.1 to 50 mg/L. In some embodiments, the auxin in the budding medium is at a concentration of 0 to 10 mg/L. In some embodiments, the cytokinin in the budding medium comprises less than 3 mg/L BAP. In some embodiments, the cytokinin in the budding medium comprises 1 mg/L BAP.

In some embodiments, the budding medium comprises half-strength MS macronutrients, full-strength micronutrients, ascorbic acid, citric acid, L-cysteine, reduced glutathione, glutamine, putrescine, ferrous sulfate, BAP, GA₃, IBA, and PPM™. In some embodiments, ascorbic acid is at a concentration in the range of 0 to 500 mg/L. In some embodiments, ascorbic acid is at a concentration in the range of 50 to 150 mg/L. In some embodiments, citric acid is at a concentration in the range of 0 to 500 mg/L. In some embodiments, citric acid is at a concentration in the range of 100 to 200 mg/L. In some embodiments, L-cysteine is at a concentration in the range of 0 to 500 mg/L. In some embodiments, L-cysteine is at a concentration in the range of 50 to 150 mg/L. In some embodiments, reduced glutathione is at a concentration in the range of 0 to 200 mg/L. In some embodiments, reduced glutathione is at a concentration in the range of 25 to 35 mg/L. In some embodiments, glutamine is at a concentration in the range of 0 to 500 mg/L. In some embodiments, glutamine is at a concentration in the range of 250 to 350 mg/L. In some embodiments, putrescine is at a concentration in the range of 0.1 to 10 mM. In some embodiments, putrescine is at a concentration in the range of 0.5 to 1.5 mM. In some embodiments, ferrous sulfate is at a concentration in the range of 0 to 250 mg/L. In some embodiments, ferrous sulfate is at a concentration in the range of 100 to 150 mg/L. In some embodiments, BAP is at a concentration in the range of 0.1 to 50 mg/L. In some embodiments, BAP is at a concentration in the range of 0.5 to 1.5 mg/L. In some embodiments, GA₃ is at a concentration in the range of 0 to 40 mg/L. In some embodiments, GA₃ is at a concentration in the range of 0 to 0.5 mg/L. In some embodiments, IBA is at a concentration in the range of 0 to 10 mg/L. In some embodiments, IBA is at a concentration in the range of 0 to 0.5 mg/L. In some embodiments, PPM™ Mis at a concentration in the range of 0 to 10 mL/L. In some embodiments, PPM™ is at a concentration in the range of 0 to 0.5 mL/L.

In some embodiments, the budding medium comprises half-strength MS macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, 125 mg/L ferrous sulfate, 1 mg/L BAP, 0.1 mg/L GA₃, 0.1 mg/L IBA, and 4 mL/L PPM™.

Chlorophenol red acts as a sensitive pH indicator for detecting contaminated cultures since bacteria and fungi contamination will change the pH even before the contamination is visible, but it does not impact the growth of the shoots. In some embodiments, chlorophenol red is optionally added at a concentration of about 5 mg/L.

In some embodiments, the bud is discernible as the emergence of new growth. In some embodiments, the bud has elongated to approximately 2-3 cm after (i). Generally, a “bud” is a small lateral or terminal protuberance on the stem of a vascular plant that is derived from meristem tissue and may develop into a flower, leaf, or shoot.

### C. Shoot Multiplication

In some embodiments, the cytokinin in the shoot multiplication medium is at a concentration in the range of 0.1 to 50 mg/L. In some embodiments, the auxin in the shoot multiplication medium is at a concentration of 0 to 10 mg/L. In some embodiments, the gibberellin in shoot multiplication medium comprises gibberellic acid (GA₃). In some embodiments, the auxin in shoot multiplication medium comprises indole-3-butyric acid (IBA). In some embodiments, the shoot multiplication medium comprises DKW medium, a sugar, putrescine, glutamine, BAP, GA₃, and IBA. In some embodiments, the sugar is sucrose, glucose, fructose, or maltose. In some embodiments, the sugar is sucrose. In some embodiments, the sugar is at a concentration in the range of 10 to 50 g/L. In some embodiments, the sugar is at a concentration in the range of 25 to 35 g/L. In some embodiments, putrescine is at a concentration in the range of 0.1 to 10 mM. In some embodiments, putrescine is at a concentration in the range of 0.5 to 1.5 mM. In some embodiments, glutamine is at a concentration in the range of 0 to 500 mg/L. In some embodiments, glutamine is at a concentration in the range of 250 to 350 mg/L. In some embodiments, BAP is at a concentration in the range of 0.1 to 50 mg/L. In some embodiments, BAP is at a concentration in the range of 0.5 to 1.5 mg/L. In some embodiments, GA₃ is at a concentration in the range of 0 to 40 mg/L. In some embodiments, GA₃ is at a concentration in the range of 0 to 0.5 mg/L. In some embodiments, IBA is at a concentration in the range of 0 to 10 mg/L. In some embodiments, IBA is at a concentration in the range of 0 to 0.5 mg/L.

In some embodiments, the shoot multiplication medium comprises DKW medium, 20 g/L sucrose, 1 mM putrescine, 300 mg/L glutamine, 1 mg/L BAP, 0.1 mg/L GA₃, and 0.1 mg/L IBA.

In some embodiments, the shoot cutting has at least three offshoots after (ii). In some embodiments, the budded shoot has four offshoots after step (ii), five offshoots after step (ii), or six offshoots after step (ii). An “offshoot” is a shoot or branch growing from the main stem of a plant.

### D. Shoot Elongation

In some embodiments, the gibberellin in the shoot elongation medium comprises GA₃. In some embodiments, the auxin in the shoot elongation medium comprises IBA. In some embodiments, the cytokinin in the shoot elongation medium comprises BAP. In some embodiments, the cytokinin in the shoot elongation medium comprises mT. In some embodiments, the shoot elongation medium comprises 3 mg/L mT.

In some embodiments, the shoot elongation medium comprises DKW medium, a sugar, putrescine, glutamine, BAP or mT, GA₃, and IBA. In some embodiments, the sugar is sucrose, glucose, fructose, or maltose. In some embodiments, the sugar is sucrose. In some embodiments, the sugar is at a concentration in the range of 10 to 50 g/L. In some embodiments, the sugar is at a concentration in the range of 25 to 35 g/L. In some embodiments, putrescine is at a concentration in the range of 0.1 to 10 mM. In some embodiments, putrescine is at a concentration in the range of 0.5 to 1.5 mM. In some embodiments, glutamine is at a concentration in the range of 0 to 500 mg/L. In some embodiments, glutamine is at a concentration in the range of 250 to 350 mg/L. In some embodiments, BAP is at a concentration in the range of 0.1 to 50 mg/L. In some embodiments, BAP is at a concentration of 0 to 0.5 mg/L. In some embodiments, mT is at a concentration in the range of 0.1 to 10 mg/L. In some embodiments, mT is at a concentration in the range of 2.5 to 3.5 mg/L. In some embodiments, GA₃ is at a concentration in the range of 0 to 40 mg/L. In some embodiments, GA₃ is at a concentration in the range of 0 to 0.5 mg/L. In some embodiments, IBA is at a concentration in the range of 0 to 10 mg/L. In some embodiments, IBA is at a concentration in the range of 0 to 0.5 mg/L.

In some embodiments, the shoot elongation medium comprises DKW medium, 20 g/L sucrose, 1 mM putrescine, 300 mg/L glutamine, 0.1 mg/L BAP or 3 mg/L mT, 0.1 mg/L GA₃, and 0.1 mg/L IBA.

### E. Growth in Optimized Culture Media

In some embodiments, the method further comprises: (iv) transferring a shoot cutting with at least three offshoots after (iii) into optimized culture medium comprising DKW medium and a gibberellin, and an auxin, and a cytokinin mT. In some embodiments, the gibberellin in optimized culture medium comprises GA₃. In some embodiments, the auxin in optimized culture medium comprises IBA. In some embodiments, the cytokinin mT in the optimized culture medium comprises 3 mg/L mT.

In some embodiments, optimized culture medium comprises DKW medium, sucrose, mT, GA₃, IBA, calcium gluconate, putrescine, glutamine, sodium thiosulfate and silver nitrate. In some embodiments, the sugar is sucrose, glucose, fructose, or maltose. In some embodiments, the sugar is sucrose. In some embodiments, the sugar is at a concentration in the range of 10 to 50 g/L. In some embodiments, the sugar is at a concentration in the range of 25 to 35 g/L. In some embodiments, mT is at a concentration in the range of 0.1 to 10 mg/L. In some embodiments, mT is at a concentration in the range of 2.5 to 3.5 mg/L. In some embodiments, GA₃ is at a concentration in the range of 0 to 50 mg/L. In some embodiments, GA₃ is at a concentration in the range of 0 to 0.5 mg/L. In some embodiments, IBA is at a concentration in the range of 0 to 1 mg/L. In some embodiments, IBA is at a concentration in the range of 0 to 0.5 mg/L. In some embodiments, calcium gluconate is at a concentration in the range of 0.1 to 10 g/L. In some embodiments, calcium gluconate is at a concentration in the range of 0.8 to 1.8 g/L. In some embodiments, putrescine is at a concentration in the range of 0.1 to 10 mM. In some embodiments, putrescine is at a concentration in the range of 0.5 to 1.5 mM. In some embodiments, glutamine is at a concentration in the range of 0 to 500 mg/L. In some embodiments, glutamine is at a concentration in the range of 250 to 350 mg/L. In some embodiments, sodium thiosulfate and sodium nitrate are at a concentration in the range of 5 to 20 mM. In some embodiments, sodium thiosulfate and sodium nitrate are at a concentration in the range of 10 to 15 mM.

In some embodiments, optimized culture medium comprises DKW medium, 30 g/L sucrose, 3 mg/L mT, 0.1 mg/L GA₃, 1 mg/L IBA, 1.3 g/L calcium gluconate, 1 mM putrescine, 300 mg/L glutamine, and 1 mL/L of 12 mM sodium thiosulfate and silver nitrate.

In some embodiments, the shoot cutting is at least 2 cm longer after (iv) than it was before (iv). In some embodiments, the shoot cutting is at least 4 cm longer after (iv) than it was before (iv). In some embodiments, the shoot cutting is 2-2.5 cm longer, 2.5-3 cm longer, 3-3.5 cm longer, 3.5-4 cm longer, 4-4.5 cm longer, or 4.4-5 cm longer after (iv) than it was before (iv).

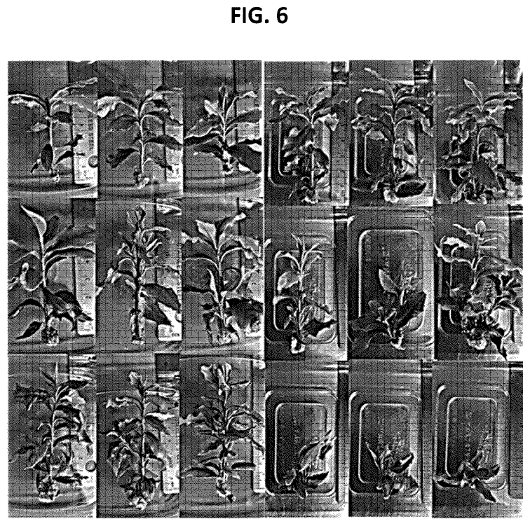

The shoot cutting grows into a juvenile plant once the shooting cutting is at least two inches tall (e.g., at least two, three, four, five, six, seven, eight, nine, or ten inches tall), with at least five (e.g., at least five, six, seven, eight, nine, or ten) leaves. A juvenile plant does not respond to flower inductive signal and does not produce flowers, fruits, or seeds. Leaves on juvenile plants are often smaller and thinner than adult plants. In a juvenile plant, shoot and leaf growths are often much more rapid than in mature tissue, as energy of the juvenile plant is focused on increasing biomass as opposed to reproduction. FIG. 6 shows some photographs of juvenile plants. In some embodiments of the method described herein, upon the shoot cutting in step (iv) of the method develops into a juvenile plant that is at least two inches tall (e.g., at least two, three, four, five, six, seven, eight, nine, or ten inches tall), has at least five leaves (e.g., at least five, six, seven, eight, nine, or ten leaves), and/or has at least one offshoot (e.g., at least one, two, three, four, or five offshoots), a step (v) of harvesting a top bud and a first set of leaves (e.g., at least two, three, four, five, six, seven, eight, nine, or ten leaves) from each offshoot of the plant is included.

In some embodiments, the step (v) of harvesting is performed between 6 and 14 weeks (e.g., between 6 and 13 weeks, between 6 and 12 weeks, between 6 and 11 weeks, between 6 and 10 weeks, between 6 and 9 weeks, between 6 and 8 weeks, between 6 and 7 weeks, between 7 and 14 weeks, between 8 and 14 weeks, between 9 and 14 weeks, between 10 and 14 weeks, between 11 and 14 weeks, between 12 and 14 weeks, between 13 and 14 weeks) after step (i) of the method is performed. In some embodiments, the step (v) of harvesting is performed between 8 and 12 weeks (e.g., between 9 and 12 weeks, between 10 and 12 weeks, between 11 and 12 weeks, between 8 and 11 weeks, between 8 and 10 weeks, between 8 and 9 weeks) after step (i) of the method is performed. In some embodiments, the step (v) of harvesting is performed 8, 9, 10, 11, or 12 weeks after step (i) of the method is performed.

In some embodiments, the step (v) of harvesting is performed once the shoot cutting grows into a juvenile plant (i.e., a juvenile plant that is at least two inches tall (e.g., at least two, three, four, five, six, seven, eight, nine, or ten inches tall), has at least five leaves (e.g., at least five, six, seven, eight, nine, or ten leaves), and/or has at least one offshoot (e.g., at least one, two, three, four, or five offshoots)).

In some embodiments, the step (v) of harvesting is performed at any time point after the plant grows into a juvenile plant or a full grown mature plant. In some embodiments, the step (v) of harvesting is performed when the plant is at least two inches tall (e.g., at least two, three, four, five, six, seven, eight, nine, or ten inches tall), has at least five leaves (e.g., at least five, six, seven, eight, nine, or ten leaves), and/or has at least one offshoot (e.g., at least one, two, three, four, or five offshoots). In some embodiment, the step (v) of harvesting is performed when the plant is at least eight inches tall (e.g., at least eight, nine, or ten inches tall), has at least ten leaves (e.g., at least ten, twelve, thirteen, fourteen, fifteen, sixteen, seventeen, eighteen, nineteen, or twenty leaves), and/or has at least two offshoots (e.g., at least two, three, four, or five offshoots).

In some embodiments, all of the leaves in an offshoot of the plant are harvested. In some embodiments, all of the leaves in all of the offshoots of the plant are harvested. In some embodiments, all of the leaves in the plant are harvested, and what remains of the plant (e.g., plant stem) is discarded.

In some embodiments, all of the leaves and buds are harvested in step (v), leaving remaining only the stem(s) of the juvenile plant, which may be used in a new cycle of the cultivating method of this invention, starting again from step (i). In some embodiments, for example, the processed dry weight of the leaves is about 25% of the wet weight of the leaves. In a situation where the plant is continuously harvested and then allowed to regrow the leaves, in some embodiments, the step (v) of harvesting is performed between 8 and 12 weeks (e.g., between 9 and 12 weeks, between 10 and 12 weeks, between 11 and 12 weeks, between 8 and 11 weeks, between 8 and 10 weeks, between 8 and 9 weeks) after step (i) of the method is performed. In some embodiments, the step (v) of harvesting is performed 8, 9, 10, 11, or 12 weeks after step (i) of the method is performed. In another example, if a large amount of leaves or a high dry weight amount of the leaves is needed, one may choose to wait until the plant has grown larger and become more mature with a high number of leaves ready to harvest.

In certain embodiments, the time between the step of placing the base of a shoot cutting from the Camellia plant in the budding medium to harvesting the leaves of the plant (i.e., from step (i) to step (v)) is about five months. As described herein, by using a series of media formulations for propagating the plant which comprise gibberellic acid (GA₃), indole-3-butyric acid (IBA), and at least one of benzylaminopurine (BAP) and meta-Topolin (mT), the method described herein provides a continuous cultivating process, which is suitable to be carried out indoors under controlled environment to rapidly generate tea plants from shoot cuttings and is able to reduce the time to harvest from about 7 years to about 5 months. Because the novel tea plant cultivating method of this invention is performed under entirely controlled and artificial conditions, it is possible for the first time ever to ensure the consistent production of tea leaves not only in quantity but also in quality, in terms of achieving the desired composition and flavor of the tea products. Furthermore, the presently described plant cultivating method does not require any pesticides.

In some embodiments, the method of propagating further comprises rooting the shoot cutting before placing it into soil.

It is understood that a shoot cutting from a Camellia plant that has any attributes described at the end of (i), (ii), or (iii) could be used in any of the subsequent steps described herein for propagation by this method.

### F. Timing of Transfer Between Media

In some embodiments, the sufficient length of time in (i) and/or (ii) is at least two intervals, wherein each interval is about 4 weeks. In some embodiments, the sufficient length of time is one interval, two intervals, three intervals, four intervals, five intervals, six intervals, seven intervals, eight intervals, nine intervals, or ten intervals. In some embodiments, each interval is 1 week, 2 weeks, 3 weeks, 4 weeks, 5 weeks, 6 weeks, 7 weeks, 8 weeks, 9 weeks, 10 weeks, 11 weeks, or 12 weeks. In some embodiments, the shoot cutting is placed in fresh medium after each interval.

In some embodiments, the shoot cutting with three shoots is sub-cultured three times at an interval of 4 weeks into fresh optimized culture medium. In some embodiments, the shoot cutting with four shoots is sub-cultured four times, five times, six times, seven times, or eight times. In some embodiments, the interval is between 4 weeks and 12 weeks long. In some embodiments, the interval is 5 weeks, 6 weeks, 7 weeks, 8 weeks, 9 weeks, 10 weeks, 11 weeks, or 12 weeks.

### G. Camellia Varieties

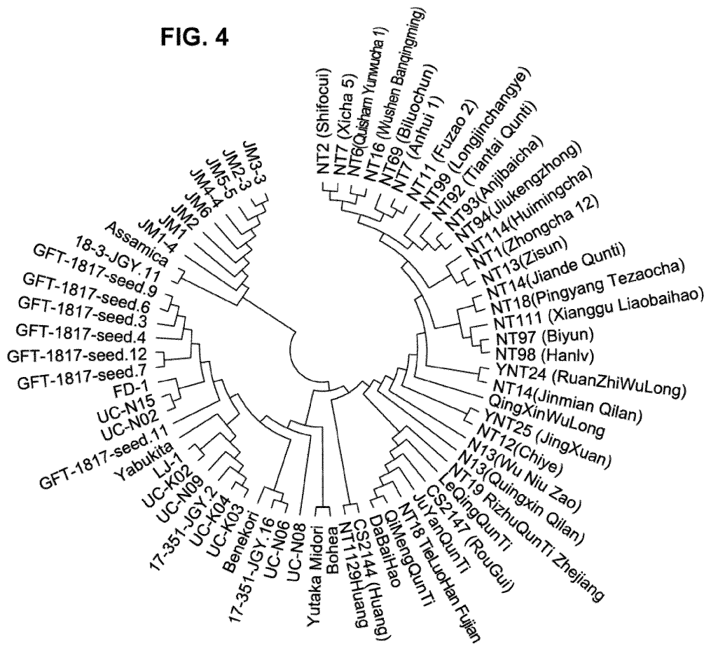

In some embodiments, the Camellia plant is a tea plant (Camellia sinensis). Camellia sinensis grows as an evergreen shrub or small tree whose leaves and buds are used to produce popular varieties of tea. Depending on the tea plant variety and processing method, these include white tea, yellow tea, green tea, oolong, dark tea (e.g., pu-erh tea), and black tea. In some embodiments, the tea plant is one of the plant varieties listed in FIG. 4. In some embodiments, the tea plant is one of Benikori, Bohea, UC-K02, UC-K03, UC-K04, UC-N02, UC-N06, UC-N08, UC-N09, UC-N15, Yabukita, and Yutaka Midori plant varieties. In some embodiments, the tea plant variety is UC-K03 or UC-K04.

### Kits for Propagating a Camellia Plant

In a second aspect, the present invention provides a kit for propagation of a tea plant. In some embodiments, the kit comprises a container which is compartmentalized for holding the various elements of the budding medium, shoot multiplication medium, and shoot elongation medium. Preferably, the kit comprises a container (e.g., a vial) which holds at least one component of the media of the invention, wherein each medium is produced by mixing the medium components set forth herein. The kit typically contains the chemical compositions of the invention, either as individual suspensions or in dehydrated form, or both, with instructions for their rehydration (if lyophilized), and use.

In some embodiments, the kit comprises a budding medium comprising MS medium with half-strength macronutrients and full-strength micronutrients or its equivalent, a gibberellin, an auxin, and a cytokinin; a shoot multiplication medium comprising DKW medium, a gibberellin, an auxin, and a cytokinin; a shoot elongation medium comprising DKW medium, a gibberellin, an auxin, and a cytokinin; and an optimized culture medium comprising DKW medium and a gibberellin, an auxin, and mT.

In some embodiments, the budding medium comprises half-strength MS macronutrients, full-strength micronutrients, ascorbic acid, citric acid, L-cysteine, reduced glutathione, glutamine, putrescine, ferrous sulfate, BAP, GA₃, IBA, and PPM™. In some embodiments, the shoot multiplication medium comprises DKW medium, a sugar, putrescine, glutamine, BAP, GA₃, and IBA. In some embodiments, the shoot elongation medium comprises DKW medium, a sugar, putrescine, glutamine, BAP or mT, GA₃, and IBA. In some embodiments, optimized culture medium comprises DKW medium, sucrose, mT, GA₃, IBA, calcium gluconate, putrescine, glutamine, sodium thiosulfate and silver nitrate.

In some embodiments, ascorbic acid is at a concentration in the range of 0 to 500 mg/L. In some embodiments, ascorbic acid is at a concentration in the range of 50 to 150 mg/L. In some embodiments, citric acid is at a concentration in the range of 0 to 500 mg/L. In some embodiments, citric acid is at a concentration in the range of 100 to 200 mg/L. In some embodiments, L-cysteine is at a concentration in the range of 0 to 500 mg/L. In some embodiments, L-cysteine is at a concentration in the range of 50 to 150 mg/L. In some embodiments, reduced glutathione is at a concentration in the range of 0 to 200 mg/L. In some embodiments, reduced glutathione is at a concentration in the range of 25 to 35 mg/L. In some embodiments, glutamine is at a concentration in the range of 0 to 500 mg/L. In some embodiments, glutamine is at a concentration in the range of 250 to 350 mg/L. In some embodiments, putrescine is at a concentration in the range of 0.1 to 10 mM. In some embodiments, putrescine is at a concentration in the range of 0.5 to 1.5 mM. In some embodiments, ferrous sulfate is at a concentration in the range of 0 to 250 mg/L. In some embodiments, ferrous sulfate is at a concentration in the range of 100 to 150 mg/L. In some embodiments, BAP is at a concentration in the range of 0.1 to 50 mg/L in the budding medium, shoot multiplication medium, and/or shoot elongation medium. In some embodiments, BAP is at a concentration in the range of 0.5 to 1.5 mg/L in the budding medium and/or shoot multiplication medium. In some embodiments, BAP is at a concentration in the range of 0 to 0.1 mg/L in the shoot elongation medium. In some embodiments, GA₃ is at a concentration in the range of 0 to 40 mg/L. In some embodiments, GA₃ is at a concentration in the range of 0 to 0.5 mg/L. In some embodiments, IBA is at a concentration in the range of 0 to 10 mg/L. In some embodiments, IBA is at a concentration in the range of 0 to 0.5 mg/L. In some embodiments, PPM™ is at a concentration in the range of 0 to 10 mL/L. In some embodiments, PPM™ is at a concentration in the range of 0 to 0.5 mL/L. In some embodiments, calcium gluconate is at a concentration in the range of 0.1 to 10 g/L. In some embodiments, calcium gluconate is at a concentration in the range of 0.8 to 1.8 g/L. In some embodiments, mT is at a concentration in the range of 0.1 to 10 mg/L. In some embodiments, mT is at a concentration in the range of 2.5 to 3.5 mg/L. In some embodiments, sodium thiosulfate and sodium nitrate are at a concentration in the range of 5 to 20 mM. In some embodiments, sodium thiosulfate and sodium nitrate are at a concentration in the range of 10 to 15 mM.

In some embodiments, the kit comprises a budding medium comprising MS medium with half-strength macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, ferrous sulfate, 1 mg/L BAP, 0.1 mg/L GA₃, 0.1 mg/L IBA, 5 mg/L chlorophenol red, and 4 mL/L PPM™, a shoot multiplication medium comprising DKW medium, 20 g/L sucrose, 1 mM putrescine, 300 mg/L glutamine, 1 mg/L BAP, 0.1 GA₃, and 0.1 mg/L IBA; a shoot elongation medium comprising DKW medium, 20 g/L sucrose, 1 mM putrescine, 300 mg/L glutamine, 0.1 mg/L BAP or 3 mg/L mT, 0.1 mg/L GA₃, and 0.1 mg/L IBA; and optimized culture medium comprising DKW medium, 30 g/L sucrose, 3 mg/L mT, 0.1 mg/L GA₃, 1 mg/L IBA, 1.3 g/L calcium gluconate, 1 mM putrescine, 300 mg/L glutamine, and 1 ml/L of 12 mM sodium thiosulfate and silver nitrate.

### Media for Propagating a Camellia Plant

By using a series of media formulations to propagate the Camellia plant, the method described herein is able to reduce the time from rooting a cutting from a Camellia plant to harvesting plant leaves (e.g., from a juvenile plant, or from a full grown mature plant) from 7 years to about 5 months or less (e.g., two, three, four, or five months). The series of media formulations are described herein.

A budding medium for propagation of a tea plant can include ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L.

A shoot multiplication medium for propagation of a tea plant can include DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L.

A shoot elongation medium for propagation of a tea plant can include DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or mT at a concentration in the range of 0.1 to 10 mg/L, GA₃ at a concentration in the range of 0 to 40 mg/L, and IBA at a concentration in the range of 0 to 10 mg/L.

An optimized culture medium for propagation of a tea plant can include DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

### IV. Examples

### Example 1. Materials and Methods

### Plant Material, Media, and Culture Conditions

Shoot cuttings approximately 4-5 cm in length were collected from three greenhouse-grown tea genotypes: Benikori, Bohea, UC-K02, UC-K03, UC-K04, UCN-06, UCN-08, UCN-09, UCN-15, Yabukita, Yutaka Midori. Shoots were rinsed under running tap water for 10 minutes then surface-sterilized for 20 minutes in a 1.2% (v/v) sodium hypochlorite solution containing 5 mL Tween 20 followed by three rinses in sterile distilled water. The base of the shoots, which were damaged by the sodium hypochlorite solution, were removed and the shoots were transferred to an agar-solidified medium.

The medium was composed of half-strength Murashige and Skoog (1962) macronutrients (MS) and full-strength Micronutrients supplemented with 100 mg/L ascorbic acid, 150 mg/L citric acid, 100 mg/L L-cysteine, 30 mg/L reduced glutathione, 300 mg/L glutamine, 1 mM putrescine, 15 mL of a 30 mM ferrous sulfate solution, 1.0 mg/L 6-benzylaminopurine (BAP), 0.1 mg/L gibberellic acid (GA₃), 0.1 mg/L indole-3-butyric acid (IBA), 5 mg/L chlorophenol red and 4 mL/L plant preservative mixture (PPM™, Plant Cell Technology Inc). Shoots were sub-cultured to a fresh medium of the same formulation every 4 weeks.

Once buds elongated to approximately 2-3 cm, shoots were removed and transferred to Driver and Kuniyuki walnut minimal organics medium (DKW) (Lloyd, and McCown, 1981) containing, 20 g/L sucrose 1 mM putrescine, 300 mg/L glutamine, 1.0 mg/L BAP, 0.1 mg/L GA₃ and 0.1 mg/L IBA. Shoots were sub-cultured to fresh media at 4-week intervals. On this medium, lateral buds expanded allowing for shoot multiplication. To stimulate shoot elongation, the cultures were transferred to DKW medium containing, 20 g/L sucrose 1 mM putrescine, 300 mg/L glutamine, 3.0 mg/L meta-Topolin (mT), 0.1 mg/L GA₃ and 0.1 mg/L IBA.

### Use of Optimized Media

Using the optimized media, 12 explants (3 blocks with 4 shoots each) were cultivated for the 12 cultivars of Camellia sinensis and were sub-cultured three times at 4-week intervals. The length of shoots and the number of explants for each cultivar were recorded after the final subculture. Optimized culture medium comprised DKW medium, 30 g/L sucrose, 3 mg/L mT, 0.1 mg/L GA₃, 1 mg/L IBA, 1.3 g/L calcium gluconate, 1 mM putrescine, 300 mg/L glutamine, and 1 ml/L of 12 mM sodium thiosulfate and silver nitrate.

### Statistical Analysis

Data collected in this study were subjected to analysis of variance (ANOVA) expressed as the mean±standard deviation (SD) and statistically analyzed using Duncan's tests (p<0.05). All experiments were measured in 3 blocks with 4 repetitions in each block (12 replicates).

### Example 2. Growth of 12 Varieties of Tea Plants in Optimized Medium

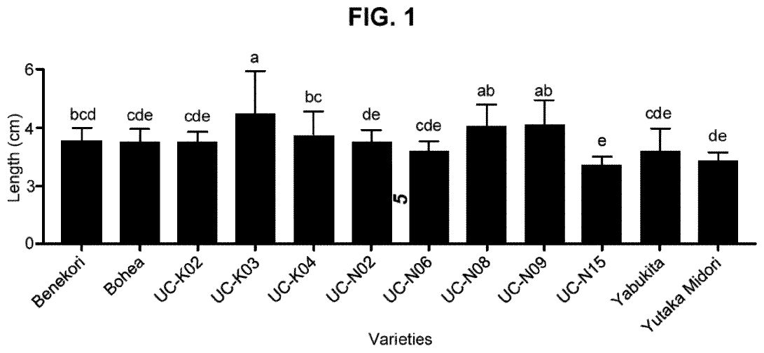

After three four-week subcultures, the plantlets of 12 different varieties of tea were evaluated for the number of shoots and elongation length, using the optimized culture medium (DKW minimal organics media supplemented with 30 g/L sucrose, 3.0 mg/L mT, 0.1 mg/L GA₃, 1.0 mg/L IBA, 1.3 g/L calcium gluconate, 1 mM putrescine, 300 mg/L glutamine, and 1.0 ml/L of 12 mM sodium thiosulfate and silver nitrate). FIG. 1 shows the elongation of the 12 varieties studied, showing an increase of length between 2.7±0.3 and 4.4±1.5 cm, where UC-N15 shows the smallest length increase and UC-K03 the largest.

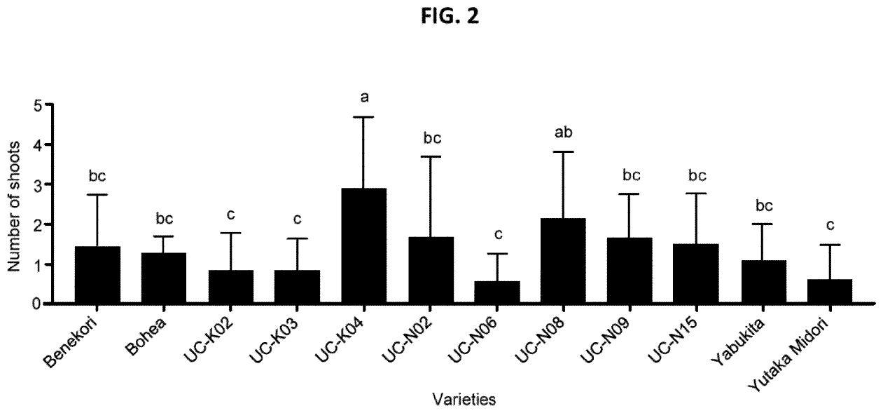

As shown in FIG. 2, the tea plant variety UC-K04 showed the highest average number of shoots, 3±1.7 shoots. Tea plant varieties UC-N06 and Yukata Midori (YM) presented the lowest values, 0.6±0.7 and 0.6±0.9, respectively.

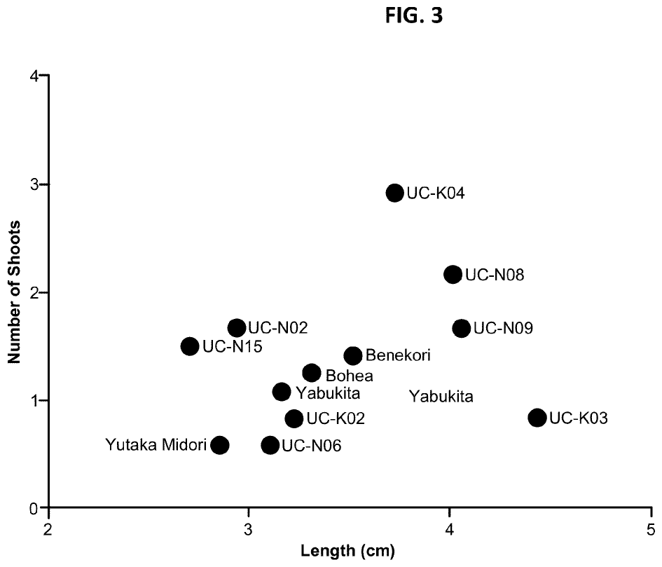

FIG. 3 show that there is no correlation between the number of shoots and elongation for the studied varieties of C. sinensis. This demonstrates that these attributes are dependent on the tea plant variety.

FIG. 4 shows the phylogenetic relatedness of 68 tea cultivars, including UC-K03 and UC-K04. Two major branches were generated in this analysis, one containing 10 cultivars and another containing 58 cultivars.

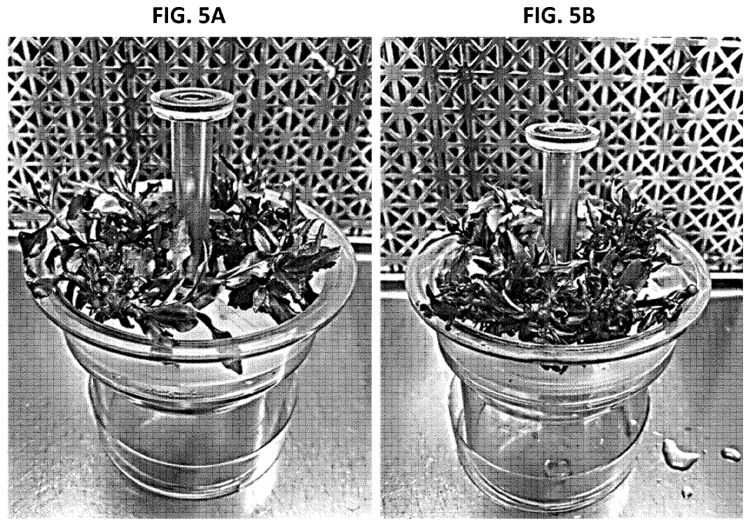

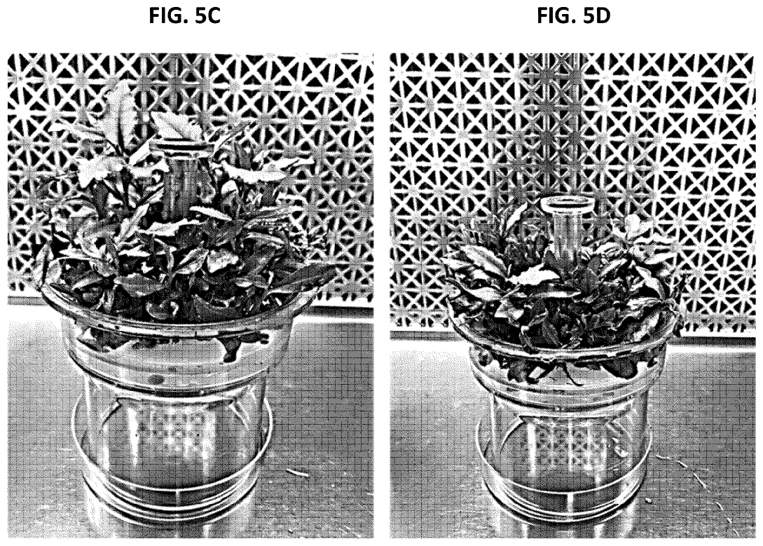

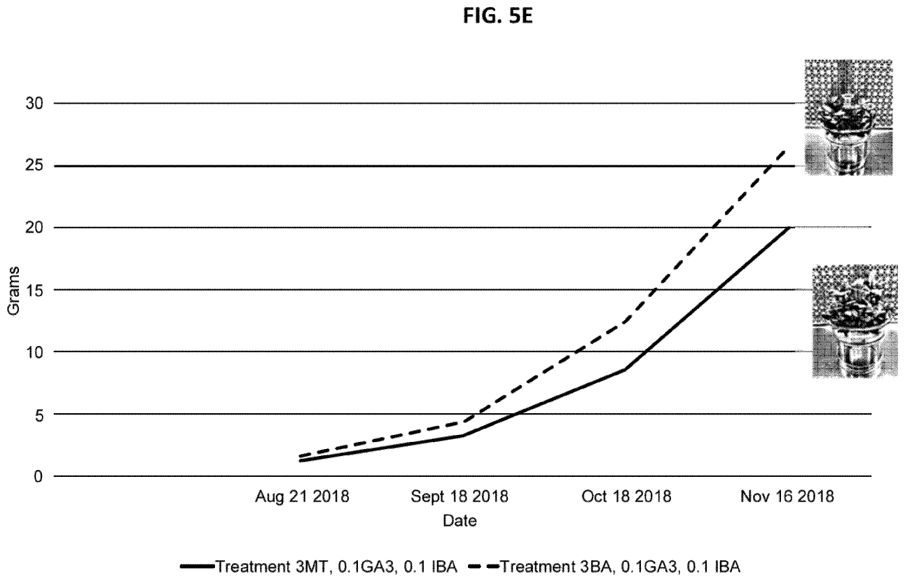

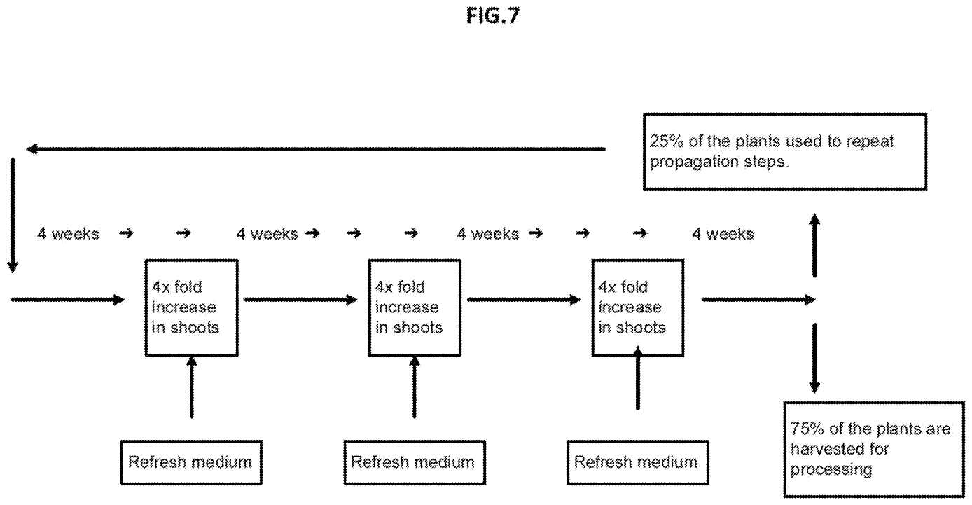

FIGS. 5A-5D show micropropagation of Benikori tea after 8 weeks and 12 weeks in RITA bioreactor on DKW minimal organics medium supplemented with 1.0 mM putrescine, 300 mg/L L-glutamine, 0.1 mg/L GA₃, 0.1 mg/L IBA and 3 mg/L mT (FIGS. 5A and 5C) or 3 mg/L BA (FIGS. 5B and 5D). Tissue was immersed in liquid medium for 2 minutes, once per day. FIG. 7 further shows an example of a schematic illustrating the use of the system to continually produce biomaterial in vitro. Several bioreactors could be run simultaneously with staggered harvest dates for continuous production.

### V. References

D'Auria et al. (2022). United States tea: A synopsis of ongoing tea research and solutions to United States tea production issues. Frontiers in plant sci. 13:934651.
Driver, J. A. and Kuniyuki, A. H. (1984). In vitro propagation of Paradox Walnut root stock. HortScience 18:506-509.
Frisch, C. H. and Camper, N. D. (1987). Effect of synthetic auxins on callus induction from tea stem tissue. Plant Cell, Tissue and Organ Culture (Vol. 8).
Gonbad, R. A. et al. (2014) Influence of Cytokinins in Combination with GA₃ on Shoot Multiplication and Elongation of Tea Clone Iran 100 (Camellia sinensis (L.) O. Kuntze) The Scientific World Journal Volume 2014, Article ID 943054, 9 pages.
Gunasekare, M. T. K. and Evans, P. K. (2000) In vitro shoot organogenesis in callus derived from stem tissue of tea (Camellia sinensis L.) Sri Lankan J. Tea Sci. 66:35-48.
Kucharska, D. et al. (2020) Application of meta-Topolin for improving micropropagation of gooseberry (Ribes grossularia). Scientia Horticulturae 272:109529.
Liang, C. et al. (2019) Optimizing Suitable Antibiotics for Bacterium Control in Micropropagation of Cherry Rootstock Using a Modified Leaf Disk Diffusion Method and E Test. Plant 8:66. https://doi.org/10.3390/plants8030066.
Murashige, T, and Skoog, F. (1962). A Revised Medium for Rapid Growth and Bio Assays with Tobacco Tissue Cultures. Physiologia Plantarum. 15:473-497.
Mukhopadyay, M. et al. (2016) Biotechnological advances in tea (Camellia sinensis [L.] O. Kuntze): a review. Plant Cell Rep. doi: 10.1007/s00299-015-1884-8.
Oseni, O. M., (2018) A Review on Plant Tissue Culture, A Technique for Propagation and Conservation of Endangered Plant Species. Intl. J. Curr. Microbiol Appl. Sci. 7:3778-86.

It is recognized that those of ordinary skill in the art may make modifications and variations in the embodiments described herein without departing from the spirit or scope of the present disclosure. All publications, patent applications, patents, figures and other non-patent publications mentioned herein are expressly incorporated by reference in their entirety.

What is claimed is:

1. A method of propagating a Camellia plant comprising:
(i) placing the base of a shoot cutting from a Camellia plant in a budding medium for a sufficient length of time until the shoot cutting has at least one bud that has elongated to at least 1 cm,
wherein the budding medium comprises Murashige and Skoog medium (MS medium) with half-strength macronutrients and full-strength micronutrients or its equivalent, a gibberellin, an auxin, and a cytokinin;
(ii) transferring the shoot cutting with at least one bud to a shoot multiplication medium for a sufficient length of time until the shoot cutting has at least two offshoots,
wherein the shoot multiplication medium comprises Driver and Kuniyuki walnut minimal organics medium (DKW medium), a gibberellin, an auxin, and a cytokinin; and
(iii) transferring the shoot cutting with at least two offshoots to a shoot elongation medium comprising DKW medium, a gibberellin, an auxin, and a cytokinin.

2. The method of claim 1, wherein the shoot cutting is 3 cm to 6 cm long before (i).

3. The method of claim 1, further comprising pretreating the shoot cutting before (i) by surface sterilizing the shoot cutting in sodium hypochlorite solution containing Tween 20 followed by rinsing in sterile distilled water and removing the base of the shoot cutting.

4. The method of claim 3, wherein the sodium hypochlorite solution is at a concentration in the range of about 0.5 to 4% (v/v), and the treatment time is 10 to 30 minutes.

5. The method of claim 3, wherein the sodium hypochlorite solution is at a concentration in the range of about 0.7 to 1.7% (v/v), and the treatment time is 15 to 20 minutes.

6. The method of claim 1, wherein the gibberellin in the budding medium comprises gibberellic acid (GA₃).

7. The method of claim 1, wherein the auxin in the budding medium comprises indole-3-butyric acid (IBA).

8. The method of claim 1, wherein the cytokinin in the budding medium comprises less than 3 mg/L benzylaminopurine (BAP).

9. The method of claim 1, wherein the cytokinin in the budding medium comprises 1 mg/L BAP.

10. The method of claim 1, wherein the budding medium comprises ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L.

11. The method of claim 1, wherein the budding medium comprises ascorbic acid at a concentration in the range of 50 to 150 mg/L, citric acid at a concentration in the range of 100 to 200 mg/L, L-cysteine at a concentration in the range of 50 to 150 mg/L, reduced glutathione at a concentration in the range of 25 to 35 mg/L, glutamine at a concentration in the range of 250 to 350 mg/L, putrescine at a concentration in the range of 0.5 to 1.5 mM, ferrous sulfate at a concentration in the range of 100 to 150 mg/L, BAP at a concentration in the range of 0.5 to 1.5 mg/L, GA₃ at a concentration in the range of 0 to 0.5 mg/L, IBA at a concentration in the range of 0 to 0.5 mg/L, and PPM™ at a concentration in the range of 0 to 0.5 mL/L.

12. The method of claim 1, wherein the budding medium comprises MS medium with half-strength macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, ferrous sulfate, 1 mg/L BAP, 0.1 mg/L GA₃, 0.1 mg/L IBA, 5 mg/L chlorophenol red, and 0.1 mL/L Plant Preservation Mixture (PPM™).

13. The method of claim 1, wherein the bud is discernible as the emergence of new growth.

14. The method of claim 1, wherein the bud has elongated to approximately 2-3 cm after (i).

15. The method of claim 1, wherein the gibberellin in the shoot multiplication medium comprises GA₃.

16. The method of claim 1, wherein the auxin in the shoot multiplication medium comprises IBA.

17. The method of claim 1, wherein the cytokinin in the shoot multiplication medium comprises less than 3 mg/L BAP.

18. The method of claim 1, wherein the cytokinin in the shoot multiplication medium comprises 1 mg/L BAP.

19. The method of claim 1, wherein the shoot multiplication medium comprises DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L.

20. The method of claim 1, wherein the shoot multiplication medium comprises DKW medium, sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0.5 to 1.5 mg/L, GA₃ at a concentration in the range of 0 to 0.5 mg/L, and IBA at a concentration in the range of 0 to 0.5 mg/L.

21. The method of claim 1, wherein the shoot multiplication medium comprises DKW medium, 20 g/L sucrose, 1 mM putrescine, 300 mg/L glutamine, 1 mg/L BAP, 0.1 mg/L GA₃, and 0.1 mg/L IBA.

22. The method of claim 1, wherein the shoot cutting has at least three offshoots from lateral bud expansion after (ii).

23. The method of claim 1, wherein the gibberellin in the shoot elongation medium comprises GA₃.

24. The method of claim 1, wherein the auxin in the shoot elongation medium comprises IBA.

25. The method of claim 1, wherein the cytokinin in the shoot elongation medium comprises meta-Topolin (mT) or BAP.

26. The method of claim 1, wherein the shoot elongation medium comprises 3 mg/L mT or 0.1 mg/L BAP.

27. The method of claim 1, wherein the shoot elongation medium comprises sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or mT at a concentration in the range of 0.1 to 10 mg/L, GA₃ at a concentration in the range of 0 to 40 mg/L, IBA at a concentration in the range of 0 to 10 mg/L.

28. The method of claim 1, wherein the shoot elongation medium comprises sucrose at concentration in the range of 25 to 35 g/L, putrescine at a concentration in the range of 0.5 to 1.5 mM, glutamine at a concentration in the range of 250 to 350 mg/L, BAP at a concentration in the range of 0 to 0.5 mg/L or mT at a concentration in the range of 2.5 to 3.5 mg/L, GA₃ at a concentration in the range of 0 to 0.5 mg/L, IBA at a concentration in the range of 0 to 0.5 mg/L.

29. The method of claim 1, wherein the shoot elongation medium comprises DKW medium, 20 g/L sucrose, 1 mM putrescine, 300 mg/L glutamine, 0.1 mg/L BAP or 3 mg/L mT, 0.1 mg/L GA₃, and 0.1 mg/L IBA.

30. The method of claim 1, further comprising (iv) transferring a shoot cutting with at least three offshoots after (iii) into optimized culture medium comprising DKW medium and a gibberellin, an auxin, and a cytokinin mT.

31. The method of claim 30, wherein the gibberellin in optimized culture medium comprises GA_{3.}

32. The method of claim 30, wherein the auxin in optimized culture medium comprises IBA.

33. The method of claim 30, wherein the cytokinin mT in the optimized culture medium comprises 3 mg/L mT.

34. The method of claim 30, wherein the optimized culture medium comprises DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

35. The method of claim 30, wherein the optimized culture medium comprises DKW medium, sucrose at a concentration in the range of 25 to 35 g/L, mT is at a concentration in the range of 2.5 to 3.5 mg/L, GA₃ at a concentration in the range of 0 to 0.5 mg/L, IBA at a concentration in the range of 0 to 0.5 mg/L, calcium gluconate at a concentration in the range of 0.8 to 1.8 g/L, putrescine at a concentration in the range of 0.5 to 1.5 mM, glutamine at a concentration in the range of 250 to 350 mg/L, sodium thiosulfate and sodium nitrate at a concentration in the range of 10 to 15 mM.

36. The method of claim 30, wherein optimized culture medium comprises DKW medium, 30 g/L sucrose, 0.1 mg/L GA₃, 1 mg/L IBA, 3 mg/L mT, 1.3 g/L calcium gluconate, 1 mM putrescine, 300 mg/L glutamine, and 12 mM sodium thiosulfate and silver nitrate.

37. The method of claim 30, wherein the shoot cutting is at least 2 cm longer after (iv) than it was before (iv).

38. The method of claim 30, wherein the shoot cutting is at least 4 cm longer after (iv) than it was before (iv).

39. The method of claim 30, further comprising, upon the shoot cutting in step (iv) develops into a juvenile plant that is at least two inches tall with at least five leaves, a step (v) of harvesting a top bud and a first set of leaves from each offshoot of the plant.

40. The method of claim 39, wherein all of the leaves from the plant are harvested.

41. The method of claim 39, wherein the time between (i) and (v) is about five months.

42. The method of claim 1, further comprising rooting the shoot cutting before placing it into soil.

43. The method of claim 1, wherein the sufficient length of time in (i) and/or (ii) is at least two intervals, wherein each interval is about 4 weeks.

44. The method of claim 43, wherein the shoot cutting is placed in fresh medium after each interval.

45. The method of claim 30, wherein the shoot cutting with at least three offshoots is sub-cultured three times at an interval of 4 weeks into fresh optimized culture medium.

46. The method of claim 1, wherein the Camellia plant is a tea plant (Camellia sinensis).

47. The method of claim 46, wherein the tea plant is one of Benikori, Bohea, UC-K02, UC-K03, UC-K04, UC-N02, UC-N06, UC-N08, UC-N09, UC-N15, Yabukita, and Yutaka Midori plant varieties.

48. The method of claim 47, wherein the tea plant is UC-K03 or UC-K04.

49. A kit for propagation of a tea plant comprising:
a budding medium comprising ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L;
a shoot multiplication medium comprising DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L;
a shoot elongation medium comprising DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or mT at a concentration in the range of 0.1 to 10 mg/L, GA₃ at a concentration in the range of 0 to 40 mg/L, and IBA at a concentration in the range of 0 to 10 mg/L; and
an optimized culture medium comprising DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

50. The kit for propagation of a tea plant of claim 49, comprising:
a budding medium comprising ascorbic acid at a concentration in the range of 50 to 150 mg/L, citric acid at a concentration in the range of 100 to 200 mg/L, L-cysteine at a concentration in the range of 50 to 150 mg/L, reduced glutathione at a concentration in the range of 25 to 35 mg/L, glutamine at a concentration in the range of 250 to 350 mg/L, putrescine at a concentration in the range of 0.5 to 1.5 mM, ferrous sulfate at a concentration in the range of 100 to 150 mg/L, BAP at a concentration in the range of 0.5 to 1.5 mg/L, GA₃ at a concentration in the range of 0 to 0.5 mg/L, IBA at a concentration in the range of 0 to 0.5 mg/L, and PPM™ at a concentration in the range of 0 to 0.5 mL/L;
a shoot multiplication medium comprising sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0.5 to 1.5 mg/L, GA₃ at a concentration in the range of 0 to 0.5 mg/L, and IBA at a concentration in the range of 0 to 0.5 mg/L;
a shoot elongation medium comprising sucrose at a concentration in the range of 25 to 35 g/L, putrescine at a concentration in the range of 0.5 to 1.5 mM, glutamine at a concentration in the range of 250 to 350 mg/L, BAP at a concentration in the range of 0 to 0.5 mg/L or mT at a concentration in the range of 2.5 to 3.5 mg/L, GA₃ at a concentration in the range of 0 to 0.5 mg/L, IBA at a concentration in the range of 0 to 0.5 mg/L; and
an optimized culture medium comprising sucrose at a concentration in the range of 25 to 35 g/L, mT at a concentration in the range of 2.5 to 3.5 mg/L, GA₃ at a concentration in the range of 0 to 0.5 mg/L, IBA at a concentration in the range of 0 to 0.5 mg/L, calcium gluconate at a concentration in the range of 0.8 to 1.8 g/L, putrescine at a concentration in the range of 0.5 to 1.5 mM, glutamine at a concentration in the range of 250 to 350 mg/L, and sodium thiosulfate and sodium nitrate at a concentration in the range of 10 to 15 mM.

51. The kit for propagation of a tea plant of claim 49, comprising:
a budding medium comprising MS medium with half-strength macronutrients, full-strength micronutrients, 100 mg/L ascorbic acid, 150 mg/L citric acid, 100 mg/L L-cysteine, 30 mg/L reduced glutathione, 300 mg/L glutamine, 1 mM putrescine, ferrous sulfate, 1 mg/L BAP, 0.1 mg/L GA₃, 0.1 mg/L IBA, 5 mg/L chlorophenol red, and 4 mL/L PPM™,
a shoot multiplication medium comprising DKW medium, 20 g/L sucrose, 1 mM putrescine, 300 mg/L glutamine, 1 mg/L BAP, 0.1 GA₃, and 0.1 mg/L IBA;
a shoot elongation medium comprising DKW medium, 20 g/L sucrose, 1 mM putrescine, 300 mg/L glutamine, 0.1 mg/L BAP or 3 mg/L mT, 0.1 mg/L GA₃, and 0.1 mg/L IBA; and
an optimized culture medium comprising DKW medium, 30 g/L sucrose, 3 mg/L mT, 0.1 mg/L GA₃, 1 mg/L IBA, 1.3 g/L calcium gluconate, 1 mM putrescine, 300 mg/L glutamine, and 1 ml/L of 12 mM sodium thiosulfate and silver nitrate.

52. A composition comprising a budding medium for propagation of a tea plant, wherein the budding medium comprises ascorbic acid at a concentration in the range of 0 to 500 mg/L, citric acid at a concentration in the range of 0 to 500 mg/L, L-cysteine at a concentration in the range of 0 to 500 mg/L, reduced glutathione at a concentration in the range of 0 to 200 mg/L, glutamine at a concentration in the range of 0 to 500 mg/L, putrescine at a concentration in the range of 0.1 to 10 mM, ferrous sulfate at a concentration in the range of 0 to 250 mg/L, BAP at a concentration in the range of 0.1 to 50 mg/L, GA₃ at a concentration in the range of 0 to 40 mg/L, IBA at a concentration in the range of 0 to 10 mg/L, and PPM™ at a concentration in the range of 0 to 10 mL/L.

53. A composition comprising a shoot multiplication medium for propagation of a tea plant, wherein the shoot multiplication medium comprises DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L, GA₃ at a concentration in the range of 0 to 40 mg/L, and IBA at a concentration in the range of 0 to 10 mg/L.

54. A composition comprising a shoot elongation medium for propagation of a tea plant, wherein the shoot elongation medium comprises DKW medium, sucrose at a concentration in the range of 10 to 50 g/L, putrescine at a concentration in the range of 0.1 to 10 mM, glutamine at a concentration in the range of 0 to 500 mg/L, BAP at a concentration in the range of 0.1 to 50 mg/L or mT at a concentration in the range of 0.1 to 10 mg/L, GA₃ at a concentration in the range of 0 to 40 mg/L, and IBA at a concentration in the range of 0 to 10 mg/L.

55. A composition comprising an optimized culture medium for propagation of a tea plant, wherein the optimized culture medium comprises DKW medium, sucrose at a concentration in the range of 10 to 50 g/L, mT at a concentration in the range of 0.1 to 10 mg/L, GA₃ at a concentration in the range of 0 to 50 mg/L, IBA at a concentration in the range of 0 to 1 mg/L, calcium gluconate at a concentration in the range of 0.1 to 10 g/L, putrescine at a concentration in the range of 0.1 to 10 mM, glutamine at a concentration in the range of 0 to 500 mg/L, sodium thiosulfate and sodium nitrate at a concentration in the range of 5 to 20 mM.

56. A Camellia plant produced by the method of claim 39.

57. A Camellia leaf harvested from the plant of claim 56.
